(19) United States
(12) Patent Application Publication
(10) Pub. No.: US 2006/0100291 A1
(43) Pub. Date: May 11, 2006

(54) Antibacterial compounds

(76) Inventors: Nigel Brian Perry, Dunedin (NZ); John William Van Klink, Dunedln (NZ); Lasley Larsen, Dunedln (NZ); Rex Thomas Weavers, Dunedln (NZ)

Correspondence Address: JACOBSON HOLMAN PLLC, 400 SEVENTH STREET N.W., SUITE 600, WASHINGTON, DC, 20004, US

(21) Appl. No.: 10/498,353

(22) PCT Filed: Dec. 17, 2002
(86) PCT No.: PCT/NZ02/00278

§ 371 Date: Jun. 17, 2004

(30) Foreign Application Priority Data
Dec. 17, 2001 (NZ) 516226

(51) Int. Cl. A61K 31/12; C07C 49/603

(52) U.S. Cl. 514/690; 568/329

(57) ABSTRACT

There is an ongoing need for new antibiotics which may be effective against bacteria that are otherwise difficult to control. The present invention therefore relates to new antibacterial triketone compounds of Formula (1), or salts, metal complexes or tautomeric forms of these compounds. The compounds have potential as novel antibiotics. Thus, the invention also relates to methods of treatment or prevention of bacterial infections using the compounds, and to compositions containing them.

### TECHNICAL FIELD

This invention relates to novel compounds possessing potent antibacterial activity. In particular, the invention relates to novel triketone compounds which exhibit activity against Gram-positive bacteria, induding Methicillin Resistant Staphylococcus aureus (MRSA), Propionibacterium acnes, and Listeda monocytogenes. The invention also relates to pharmaceutical preparations containing the triketones, and to their use as antibacterial agents.

### BACKGROUND

Many of the commonly prescribed antibiotics are becoming ineffective in the treatment or prevention of bacterial infections. The number of pathogenic bacteria which have developed resistance to antibiotics is increasing and the threat of untreatable bacterial infections is growing. The incidence of so-called “super bugs” in hospitals is becoming more frequent. There is therefore an urgent need for new antibiotics which are effective against bacteria that are otherwise difficult to control.

A series of naturally occurring compounds (known as tiketones) have been identified in plants from the family Myrtaceae, in particular the genus Leptospennum.

These triketones are characterised by three ketone functional groups on a six-membered cyclic ring as depicted in Formula A. However, such compounds will typically exist in the enol tautomeric form as shown in Formula B.

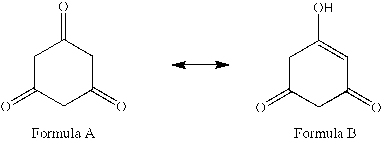

A number of triketones having further substituents on the ring structure are known. Several compounds of Formula C have been isolated from plants or have been independently synthesised. 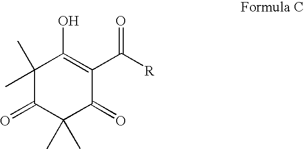
where R═CH(CH₃)₂, CH₂CH(CH₃₎₂, CH(CH₃)CH₂CH₃, CH₂CH₂Ph, or (CH₂)₄CH3.

The structure of leptospermone (R═CH₂CH(CH₃)₂) was confirmed by Briggs, eL al. (J. Chem. Soc., 1945, 706). Its extraction from steam distilled L. scopatium oil was previously described by Gardner (J. Soc. Chem. Ind., 1925, 44, 528T). Grandiflorone (R═CH₂CH₂Ph) has been isolated from L. flavescens var. grandiflora and from L. lanigerum (Hellyer and Pinhey, J. Chem. Soc. 1966, 1497). Isoleptospermone (R═CH(CH₃)CH₂CH₃) and papuanone (R═(CH₂)₄CH₃) have been isolated from L. scoparium and Corymbia dallachiana respectively (van Klink et al., J. Nat. Prod., 1999, 62, 487).

In addition, antimicrobial activity has been reported for mixtures of naturally occurring triketones found in steam distilled L. scoparium oil. For example, a mixture of flavesone (R═CH(CH₃)₂), leptospermone, and isoleptospermone obtained from L. scoparum has been reported to have a minimum inhibitory concentration (MIC) of 195 μg/ml against MRSA (Porter and Wilkins, Phytochem., 1998, 50, 407). A mixture of the same triketones has been shown to exhibit in vito antibacterial activity against Gram-positive bacteria, including Enterococcus faecium, with MICs of 100 to 400 μg/ml (Christoph et al., Planta Med., 2000, 66, 556).

However, a compound of Formula C where R═CH₃ does not show antimicrobial activity (Yamaki et al., Phytotherapy Research 1994, 8, 112).

Various triketones have also been shown to exhibit other types of biological activity. For example, leptospermone and grandiflorone are known to inhibit drug metabolism enzymes (Graham et al., Biochem. Pharmacol., 1970, 19, 769; and Graham et al., Biochem. Pharmacol., 1970, 19, 759). In addition, a number of 15 triketones of Formula C are known to have herbicidal activity (Gray et al., U.S. Pat. No. 4,202,840).

Compounds of Formula D have also been shown to have herbicidal activity (EP 409350).

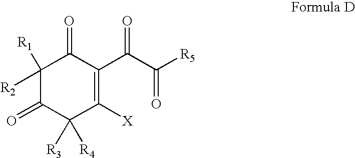

Synthesised triketones of Formula E are known to be active against Gram-positive bacteria (Lloyd et al., Antimicrobial Agents and Chemotherapy, 1988, June, 814).

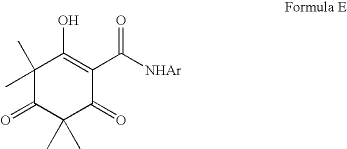

Other triketones, such as the compound of Formula F which has been isolated from the essential oil of Melaleuca cajeputi leaves, are known as sunscreens, bactericides, and fungicides (EP 613680, U.S. Pat. No. 5,411,728). Antibacterial activity was reported at concentrations of 1000 μg/ml.

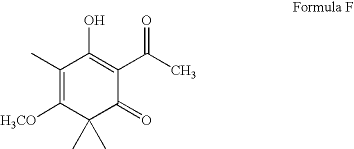

Of the known triketones of Formula C that exhibit antibacterial activity, MICs are reported to be in the order of several hundred μg/ml. For example, 100-400 μg/ml in the case of the mixture reported in Planta Med., 2000, 66, 556 above. Compounds with MICs of this order of magnitude are usually not regarded as sufficiently potent to constitute the active ingredient of a clinically useful antibiotic. Such antibiotics would typically have an MIC in the order of <10 μg/ml. For example, vancomycin has an MIC of approximately 2 μg/ml.

Other triketones have been synthesised and structurally characterised (for example compounds of Formula G (Ayras et al., Planta Med., 1981, 42, 187) but their biological properties have not been investigated.

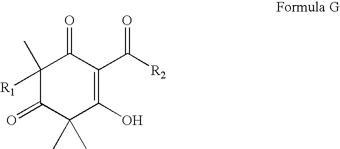

It is also known that certain hop acids (structures of α- and β-acids are given below) show antimicrobial activity. For example, EP 606599 describes oral care compositions containing tetrahydroisohumulone, tetrahydroisoadhumulone, tetrahydroisocohumulone, Rho-isohumulone, Rho-isoadhumulone, Rho-isocohumulone, lupulone, adlupulone, colupulone, hexahydrolupulone, hexahydroadlupulone and/or hexahydrocolupulone.

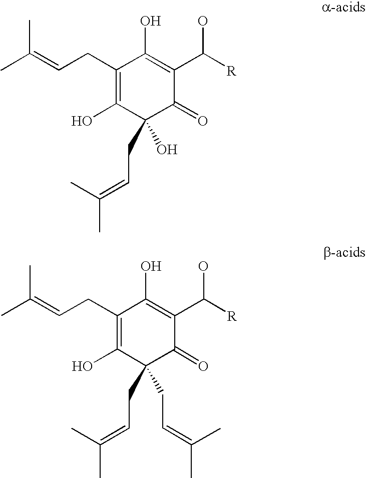

Other hop acids are known (see for example JP 07196572-A; Drewett et al., J. Inst Brew., 1970, 76, 188; Elvidge et al., J. Chem. Soc. C 1967, 19, 1839). However, the potential of these compounds as antimicrobial agents has not been investigated.

The inventors have now found that certain chemically synthesised triketones exhibit surprisingly potent antibacterial activity. The synthesised triketones therefore represent a class of compounds with enormous potential as novel antibiotics.

Accordingly, it is an object of the invention to provide novel compounds having antibacterial activity, or at least to provide a useful alternative.

### STATEMENTS OF INVENTION

In a first aspect, the Invention provides a compound of Formula (1): 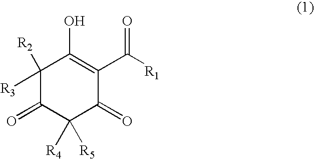
where R₁ is a group selected from alkenyl, alkynyl and C₆-C₂₀ alkyl, each of which may be substituted with one or more of the groups selected from hydroxy, halogen, amino, alkylamino, di-alkylamino, haloalkyl, nitro, cyano, —SO3H and triphenylphosphine;
or R₁ is a group selected from aryl, C₅-C₈ cycloalkyl, (C₁-C₂₀ alkyl)cydoalkyl and (C₁-C₂₀ alkyl)aryl, each of which may be substituted with one or more of the groups selected from hydroxy, halogen, amino, alkylamino, di-alkylamino, haloalkyl, nitro, cyano, —SO₃H, triphenylphosphine, alkyl, alkenyl, and alkynyl; provided that R₁ is not —CH₂CH₂phenyl; and
R₂ to R₅ are each independently alkyl, alkenyl or alkynyl groups, each of which may be substituted with one or more of the groups selected from hydroxy, halogen, amino, alkylamino, di-alkylamino, haloalkyl, nitro, cyano, —SO₃H and triphenylphosphine;
   or R₂ to R % are each independently aryl or acyl groups, each of which may be substituted with one or more of the groups selected from hydroxy, halogen, amino, alkylamino, di-alkylamino, haloalkyl, nitro, cyano, —SO₃H, triphenylphosphine, alkyl, alkenyl and alkynyl;
or a salt thereof, or a metal complex therof, or any tautomerio form thereof;
with the proviso that compounds of formulae (i) and (ii) are excluded

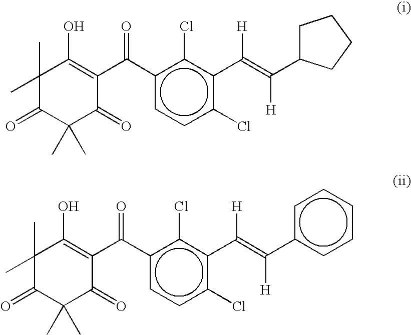

Preferably R₂ to R₅ are each independently allyl or alkenyl groups. More preferably R₂ to R₅ are all methyl groups. It is also preferred that one or more of R₂ to R₅ is a prenyl group

It is further preferred that R₁ is C₆-C₂₀ straight chain alkyl or (C₁-C₂₀alkyl)phenyl. More preferably R₁ is C₁₀-C₁₆ straight chain alkyl.

Preferred compounds of the invention are:
5-hydroxy-4-(1-exododecyl)-2,2,6,6-tetramethyl-4-cyclohexene-1,3-dione;
5-hydroxy-4-(1-oxodecyl)-2,2,6,6-tetramethyl-4-cyclohexene-1,3-dione;
5-hydroxy-4-(1-oxohexadecyl)-2,2,6,6-tetramethyl-4-cyclohexene-1,3-dione;
5-hydroxy-4-(1-oxomethylcyclohexyl)-2,2,6,6-tetrametylclohexene-1,3-dione;
5-hydroxy-4-(1-oxopropylcyclohexl)-2,2,6,6-tetramethyl-4-cyclohexene-1,3dione;
5-hydroxy-4-(1-oxododecyl)-2,2,2,6,-tetramethyl-4-cyclohexene-1,3-dione; or
5-hydroxy-4-(1-oxododecyl-2-prenyl-2,6,6-trimethyl-4-cyclohexene-1,3-dione.

In a second aspect, the Invention provides a pharmaceutical composition containing a compound as defined above, together with a pharmaceutically acceptable camier. The invention also provides an antibactenal agent containing a compound as defined above, In another aspect, the invention provides the use of a compound as defined above in the manufacture of an antibacterial agent.

There is also provided a process for preparing a compound as defined above, including the steps of:
(a) reacting phloroglucinol with a carboxylic acid of the formula R₁—COOH, or equivalent acid halide, nitrite or anhydride, where R₁ is as defined above, to give a substituted phloroglucinol of the formula 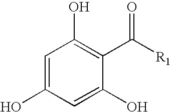
(b) reacting the substituted phloroglucinol with one or more compounds of the formula AX where A is R₂, R₃, R₄ or R₅, each as defined above, and X is a halogen atom.

In a final aspect, the invention provides a method of treatment or prevention of a bacterial infection in a human or other animal comprising administering to the human or other animal a therapeutically effective amount of a compound as defined above.

The invention therefore provides methods of treatment or prevention of bacterial infections, where the bacterial infection is caused by Staphylococcus aureus, Methicillin Resistant Staphylococcus aureus, Erythromycin Resistant Staphylococcus aureus, Mupirocin Resistant Staphylococcus aureus, Oxacillin/Gentamicin Resistant Staphylococcus aureus, Vancomycin/Oxacillin Resistant Staphylococcus aureus, Bacillus subtilis, Enterococcus faecalis, Gentamicin Resistant Enterococcus faecalis, Vancomycin Resistant Enterococcus faecalis, or Ampicillin Resistant Enterococcus faecalis.

### DETAILED DESCRIPTION

The term “C₆-C₂₀ alkyl” means a straight chain or branched saturated hydrocarbon radical having from 6 to 20 carbon atoms and includes, for example, decyl, dodecyl, hexadecyl, and the like.

The term “C₅-C₈ cycloalkyl” means a cyclic saturated hydrocarbon radical having from 5 to 8 carbon atoms, and includes cyclohexyl and the like. “Cycoalkyl” means a cyclic saturated hydrocarbon radical.

The term (C₁-C₂₀ alkylicyloalkyl” means a straight chain or branched saturated hydrocarbon radical attached to a cyclic saturated hydrocarbon radical.

The terms “alkenyl” and “alkynyl” mean straight chain or branched hydrocarbon radicals, where any alkenyl group has one or more carbonrcarbon double bonds and where any alkynyl group has one or more carbon-carbon triple bonds.

The term “aryl” means an aromatic radical, such as phenyl, naphthyl, etc.

The term “acyl” includes alkanoyl groups such as formyl, acetyl, propanoyl, etc.

The salts of the compounds of Formula (1) are intended to include salts derived from organic or inorganic bases including salts derived from amines and pyridines and metal hydroxides, carbonates and bicarbonates.

The metal complexes of Formula (1) are intended to include complexes formed with metal ions such as Fe³⁺ and Cu²⁺.

Compounds of the Formula (1) may possess one or more chiral centres. The invention therefore includes all diastereomeric, enanffomeric, and epimeric forms, as well as mixtures of them. In addition, compounds of the invention may exist as geometric isomers. The invention therefore includes all cis and trans (syn and anti), isomers as well as mixtures of them.

It will be appreciated that the arrangement of enol and carbonyl groups in compounds of the Formula (1) allows for tautomeric isomerism. It is to be appreciated that the tautomeric forms include compounds of the Formulae (1A), (1B), and (1C). 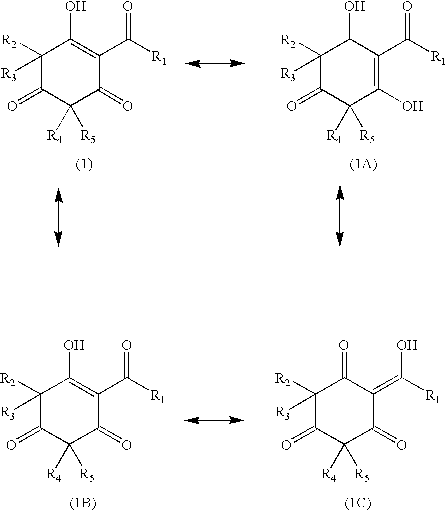
where R₁ to R₅ are as defined above.

Preferred compounds of the Invention are those where R₂ to R₅ are alkyl or alkenyl groups. Most preferably, R₂ to R₅ are all methyl groups.

Preferred compounds of the invention include those where R₁ is a C₆-C₂₀ alkyl, (C₁-C₂₀ alkyl)aryl, C₅-C₈ cycloalkyl, or (C₁-C20alkyl)cycloalkyl group. When R₁ is a C₆-C₂₀ alkyl group, it is preferred to be a C₁₀-C₁₆ alkyl group. When R₁ is (C₁-C₂₀alkyl)aryl, it is preferred to be a C₁-C₂₀ alkylphenyl, provided that R₁ is not —H₂CH₂phenyl. When R₁ is a C₅-C₈ cycloalkyl group, it is preferred to be cydohexyl. When R₁ is a (C₁-C₂₀alkyl)rydoalkyl group, it is preferred to be a (C₁-C₉alkyl)cyclohexyl group.

Preferred compounds of the invention include:
5-hydroxy-4-(1-exododecyl)-2,2,6,6-tetramethyl-4-cyclohexene-1,3-dione;
5-hydroxy-4-(1-oxodecyl)-2,2,6,6-tetramethyl-4-cyclohexene-1,3-dione;
5-hydroxy-4-(1-oxohexadecyl)-2,2,6,6-tetramethyl-4-cyclohexene-1,3-dione;
5-hydroxy-4-(1-oxomethylcyclohexyl)-2,2,6,6-tetrametylclohexene-1,3-dione;
5-hydroxy-4-(1-oxopropylcyclohexl)-2,2,6,6-tetramethyl-4-cyclohexene-1,3dione;
5-hydroxy-4-(1-oxododecyl)-2,2,2,6,-tetramethyl-4-cyclohexene-1,3-dione; or
5-hydroxy-4-(1-oxododecyl-2-prenyl-2,6,6-trimethyl-4-cyclohexene-1,3-dione.

The compounds of Formula (1) may be prepared by standard chemical synthesis methods.

A preferred method includes reacting phloroglucinol with a carboxylic acid in the presence of aluminium chloride and phosphorous oxychloride to acylate an available carbon atom of the aromatic ring of phloroglucinol. Although diacylation can occur, the monoacylated compound is typically the major product

In the case of the tetramethyl compounds, the mono-C-acylated phloroglucinol is then reacted in the presence of a strong base, such as sodium methoxide, with methyl iodide. The tetraalkylated compound is generally recovered as the major product of the reaction.

For the tetraethyl compounds, the monoacylated phloroglucinol is reacted in the presence of a strong base, such as sodium methoxide, with ethyl iodide.

In the case of the monoprenyl derivatives, the monoacylated phloroglucinol is reacted with prenyl bromide to give a monoprenyl-phloroglucinol. This monoprenyl-phloroglucinol may be then further reacted with methyl iodide to produce trimethyl-monoprenylcompounds.

A preferred method for preparing Cu(II) complexes of the compounds includes refluxing a mixture of the triketone and cupric acetate in methanol, followed by extraction from ether and crystallisation from methanol. The compounds of the invention exhibit biological activity against a range of Gram-positive bacteria. The compounds are therefore considered to be useful in the treatment or prevention of a range of bacterial infections. Such infections include those caused by Staphylococcus aureus (including MRSA), S. epidermis, S. saprophyticus, Enterococcus faecalis (including vancomycin resistant strains), Listeria monocytogenes, and Propionibacterium acnes, Streptococcus mutans, Streptococcus ovalis, and Actinomyces naeslundii.

The inventors have shown that the compounds of the invention exhibit activity against a number of different pathogens. Table 1 (Example 8) shows the activities of the compounds of Examples 1-7 against MRSA and Bacillus subtilis.

All of the compounds showed activity against MRSA. Compound 1 exhibited a MIC of between 0.5 and 1.0 μg/ml. The remaining compounds exhibited MICs of between approximately I and 8 μg/ml. Some of the compounds also exhibited activity against Bacillus subtilis in a disc diffusion assay.

Furthermore, the inventors have found that the compounds of the invention show significant activity against a number of resistant bacteria. Table 2 (Example 9) shows the activity of the compound of Example 1 against gentamicin resistant, vancomycin resistant and ampicillin resistant Enterococcus faecalis, and against erythromyin resistant, mupirocin resistant, oxacillin resistant and vancomycin/oxacillin resistant S. aureus. Thus, the compounds are expected to be useful for the treatment of diseases associated with these resistant bacteria.

Acute toxicity tests (Example 10) show that the compound of Example I is not toxic to mice at the levels administered intraperitoneally.

The amount of active ingredient to be administered may vary widely according to the nature of the bacterial infection and the nature of the patient A typical dosage for an adult human is likely to be in the range of 0.1 to 1000 milligrams when administered orally. The active ingredient will be administered with one or more conventional pharmaceutical carriers. Administration may be oral, topical, or by injection, or by any other known means of administration.

The compounds of the invention can be formulated into solid or liquid preparations, for example tablets, capsules, powders, solutions, suspensions, and dispersions. Certain liquid preparations may be effective as a mouth rinse. The carrier may be one or more substances acting as diluents, flavouring agents, solubilisers, lubricants, suspending agents, binders, preservatives, tablet disintegrating agents, or an encapsulating material.

### EXAMPLES

The invention will now be described in further detail with reference to the following examples. It is to be appreciated that the invention is not limited to these examples.

### Example 1

### Preparation of 5-hydroxy-4-(1-oxododecyl)-2,2,6,6-tetramethyl-4-cyclohexene-1,3-dione

Dry phloroglucinol (1.26 g, 10 mmol) was added to a stirred solution of dry AlCl₃ (4 g) in POCl₃ (15 ml) and the solution stirred under nitrogen for 30 min. Dodecanoic acid (10 mmol) was added slowly with stirring at 0° C. then the mixture stirred for a further 4 h at 0° C., and then for 40 h at 6° C. The mixture was poured onto ice (50 g) then extracted into ethyl acetate, washed with saturated sodium bicarbonate solution, dried and evaporated under vacuum to give the crude product Purification by column chromatography over silica gel eluting with dichloromethane with increasing amounts of ethyl acetate gave first diacylated phlorogenols then the mono-C-acyl phlorogenol. The non-optimised yield for the mono-C-acyl phlorogenol was determined to be 25%.

Sodium metal (0.3 g) was slowly added to methanol (5 ml) to form a solution. To this was added the mono-acylated phloroglucinol (200 mg) followed by methyl iodide (5 ml). The solution was refluxed under nitrogen for 3 h. Addition of HCl (1M) until just acidic, followed by extraction into ethyl acetate gave the crude product Purification by column chromatography over silica gel eluting with hexane with increasing amounts of dichloromethane gave the triketone. 5-Hydroxy-4-(1-oxododecyl)-2,2,6,6-tetramethyl-4-cylohexene-1,3-dione was obtained as a pale yellow oil (non-optimised yield 80%): found C 72.52% H 9.81%, calcd for C₂₂H₃₆O₄, C 72.48% H 9.95%; Si gel TLC R_{F} 0.45 (9:1 Hex: EtOAc, UV visualisafion); UV (hexane) λ max (log ε) 277 (4.23), 236 (3.86) nm; IR (film) υ max 2924, 1721, 1670, 1559, 1465, 1381, 1048 cm⁻¹; EIMS 70 eV m/z (rel. int.) 364 [M]⁺ (95), 349 (23), 346 (20), 331 (10), 294 (70), 237 (93), 224 (96), 209 (40), 196 (35), 181 (18), 167 (40), 154 (58), 139 (25), 113 (31), 96 (100), 81 (44), 70 (60), 69 (62), 55 (71); ¹³C NMR (CDCl₃) 210.0, 204.7, 199.0, 196.7, 109.1, 56.9, 52.1, 39.2, 32.0, 31.9, 29.6, 29.5, 29.4, 25.2, 24.3, 23.9, 22.7, 14.1 ppm; ¹H NMR (CDCl₃) 18.37 (1H s, OH), 2.97 (2H, t, J 8 Hz), 1.65 (2H, m), 1.46 (6H, s), 1.37 (6H, s), 1.26 (16H, m), 0.88 (3H, t, J 7 Hz) ppm.

### Example 2

### Preparation of 5-hydroxy-4-(1-oxodecyl)-2,2,6,6,-tetramethyl-4-cyclohexene-1,3-dione

The above compound was prepared in the same manner as the compound of Example 1. The nonoptimised yield for the mono-C-acyl phlorogenol was determined to be 23%. 5-Hydroxy-4-(1-oxodecyl)2,2,6,6-tetramethyl-4-cyclohexene-1,3-dione was obtained as a colourless oil (176 mg, 73%); Si gel TLC (Hexane/Dichloromethane (50:50)), R_{F} 0.28 detection by UV light; UV (MeOH) λ max (log ε) 278 (4.0 ) and 238 (3.8) nm; IR (dry film) υ max 2927, 2855, 1723, 1672, 1666, 1581, 1564, 1552, and 1049 cm⁻¹; ¹³C NMR (CDCl₃) 210.0, 204.8, 199.1 (C-2′), 196.8 (C-6′), 109.0 (C-l′), 56.8 (C-5′), 52.1 (C-3′), 39.2 (C-2), 31.9 (C-3), 29.4, 29.3, 29.2, 25.1, 24.3 (C-3′Me's), 23.8 (C-5′Me's), 22.6, 14.1 (C-10) ppm; ¹H NMR (CDCl₃) 18.34 (1H, s, OH), 2.96 (2H, t, J 8 Hz, C(2)H₂), 1.63 (2H, m), 1.44 (6H, s, C (3′)Me₂), 1.35 (6H, s, C (5′)Me₂), 1.25 (12H, m) and 0.86 (3H, t, J 7 Hz) ppm; Mass measurement, m/z Found, 336.23116; C₂₀H₃₂O₄ requires 336.23006; EIMS (70 eV) m/z 336 (M⁺, 30%), 321 (21), 266 (62), 237 (89), 224 (63),154 (53) and 96 (100).

### Example 3

### Preparaton of 5-hydroxy-4(1-oxohexadecyl)-2,2,6,6-tetramethyl-4-cyclohexene-1,3-dione

The above compound was prepared in the same manner as the compound of Example 1. The non-optimised yield for the monoactyl phlorogenol was determined to be 16%. 5-Hydroxy4(1 -oxohexadecyl)2,2,6,6-tetramethyl-4-cyclohexene1,3-dione was obtained as a white crystalline solid (199 mg, 86%): mp 39.0° C.; Si gel TLC (Hexane/Dichloromethane (50:50)), R_{F} 0.31 detection by UV light; UV (MeOH ) λ max (log s) 278 (4.0), and 239 (3.8) nm; IR (dry film) υ max 2927, 2855, 1723, 1672, 1666, 1581, 1564, 1552, and 1049 cm⁻¹; ¹H NMR (CDCl₃) 18.34 (1H, s, OH), 2.96 (2H, t, J 8 Hz, C(2)H₂), 1.64 (2H, m), 1.44 (6H, s, C (3′)Me₂), 1.35 (6H, s, C (5′)Me₂), 1.24 (24H, m) and 0.87 (3H, t, J 7 Hz) ppm. ¹³C NMR (CDCl₃) 210.2 (C-′), 204.8 (C-1), 199.1 (C-2′), 196.8 (C-6′), 109.0 (C-1′), 56.8 (C-5′), 52.1 (C-3′), 39.2 (C-2), 31.9 (C-3), 29.7, 29.6, 29.5, 29.3, 25.1, 24.3 (C-3′Me's), 23.9 (C-5′Me's), 22.7, 14.1 (C-10) ppm; Mass measurement, m/z Found, 420.32577; C₂₆H₄₄O₄ requires 420.32396; EIMS (70 eV) m/z: 420 (Mt. 23%), 350 (32), 237 (99), 224 (79), 154 (52) and 96 (100).

### Example 4

### Preparation of 5-hydroxy-4(1-oxomethylcyclohexyl)-2,2,6,6-tetramethyl-4-cyclohexene-1,3dione

The above compound was prepared in the same manner as the compound of Example 1. The non-optimised yield for the monoactyl phlorogenol was determined to be 27%. 5-Hydroxy4-(1-oxomethylcyclohexylI2,2,6,6-tetramethyl4-cyclohexene-1,3-dione was obtained as a white crystalline solid (166 mg, 54%): mp 47.8° C.; Si gel TLC (Hexane/Dichloromethane (50:50)), R_{F} 0.23 detection by UV light; UV (MeOH) λ max (log ε) 280 (4.1), and 240 (3.8) nm; IR (dry film) υ max 2932, 2854, 1723, 1672, 1666, 1581, 1563, 1549, and 1049 cm⁻¹; ¹³C NMR (CDCl₃) 210.0 (C-4′), 207.3(C-1), 199.8 (C-2′), 196.9 (C-6′), 108.2.(C-1′), 57.0 (C-5′), 52.4 (C-3′), 45.1 (C-2), 29.3 (C-3,7), 25.8 (C-5), 25.7 (C-4,6), 24.3 (C-3′Me's), 23.9 (C-5′Me's) ppm. ¹H NMR (CDCl₃) 18.54 (1H, s, OH), 3.50 (2H, t, J 8 Hz, C(2)H), 1.80 (4H, m), 1.43 (6H, s, C (3′)Me₂), 1.38 (6H, m), 1.35 (6H, s, C (5′)Me₂) ppm; Found: C, 69.65, H, 8.10. C₁₇H₂₄O₄ requires: C, 69.86, H, 8.22. Mass measurement, m/z Found, 292.16837; C₁₇H₂₄0₄ requires 292.16746; EIMS (70 eV) m/z:: 292 (M⁺, 94%), 277 (47), 222 (56), 204 (47), and 83 (100).

### Example 5

### Preparation of 5-hydroxy-4-(1-oxopropylcyclohexl)-2,6,6-tetramethyl-4-cyclohexene-1,3-dione

The above compound was prepared in the same manner as the compound of Example 1. The non-optimised yield for the mono-C-acyl phlorogenol was determined to be 51%. 5-Hydroxy(1 -oxopropylcyclohexl)2,2,6,8tetramethyl-4-cyclohexene-1,3-dione was obtained as a yellow crystalline solid (155 mg, 58%): m.p. 45° C.; Si gel TLC (Hexane/Dichloromethane (50:50)), R_{F} 0.27 detection by UV light; UV (MeOH) λ max (log ε) 279 (4.2), and 238 (4.0) nm; IR (dry film) υ max 2924, 2852, 1723, 1672, 1666,1581, 1563, 1549 and 1049 cm⁻¹; ³C NMR (CDCl₃) 210.0 (C-4′), 205.2 (C-1), 199.0 (C-2′), 196.8 (C-6′), 109.9 (C-1′), 56.8 (C-5′), 52.1 (C-3′), 37.5 (C-2), 36.8 (C-3), 32.5 (C-4), 33.1 (C-5,9), 26.5 (C-7), 26.2 (C-6,8), 24.3 (C-3′Me's), 23.8 (C-5′Me's) ppm. ¹H NMR (CDCl₃) 18.34 (1H, s, OH), 2.98 (2H, t, J 8 Hz, C(2)H₂), 1.71 (5H, m), 1.52 (2H, m), 1.44 (6H, s, C (3′)Me₂), 1.35 (6H, s, C (5′)Me₂), 1.20 (4H, m) and 0.93 (2H, m) ppm; Found, C, 71.54, H, 8.74; C₁₉H₂₈O₄ requires C, 71.25, H, 8.75; EIMS (70 eV) m/z. 320 (M⁺, 17%), and 237 (100).

### Example 6

### Preparation of 5-hydroxy-4-(1-oxododecyl-2,2,6,6-tetraethyl-4-cyclohexene-1,3dione

Sodium metal (0.3 g) was slowly added to methanol (5 ml) to form a solution. To this was added the mono C dodecanoylated phloroglucinol (200 mg), prepared as above in Example 1, followed by ethyl iodide (5 ml). The solution was refluxed under nitrogen for 3 h. Addition of HCl (1 M) until just acidic, followed by extraction into ethyl acetate gave the crude product. Purification by column chromatography over silica gel eluting with hexane with increasing amounts of dichloromethane gave 5-hydroxy-4-(1-oxododecyl)2,2,6,6-tetraethyl-4-cyclohexene-1,3-dione as a colourless oil (109 mg, 40%). Si gel TLC (Hexane/Dichloromethane (50:50)), R_{F} 0.51 detection by UV light; UV (MeOH ) λ max (log ε) 320 sh (3.3), 281 (4.1), and 238 (3.9) nm; IR (dry film) υ max 2926, 2854, 1711, 1668, 1666, 1581, 1564, 1552, and 1068 cm⁻¹;¹³C NMR (CDCl₃) 207.8 (C-4′), 204.3 (C-1), 198.3 (C-2′), 195.7 (C-6′), 111.0 (C-1′), 65.7 (C-5′), 61.2 (C-3′), 39.3 (C-2), 31.9 (C-10), 29.7, 29.6, 29.5, 29.4, 29.3, 28.2 (C-1″, -1′″), 25.1 (C-3), 22.7 (C-11), 14.1 (C-12), 9.4 (C-2″) and 9.2 (C-2′″) ppm; ¹H NMR (CDCl₃) 18.41 (1H, s, OH), 2.98 (2H, t, J 8 Hz, C(2)H₂), 1.79 (8H, m, C(1″, 1′″)H₂), 1.63 (2H, m), 1.25 (16H, m),0.86 (3H, t, J 7 Hz, C(12)H₃), and 0.55 (12H, dt, J 10, 12 Hz, C(2″, 2′″)H₃) ppm. Mass measurement, m/z Found, 420.32390; C₂₆H₄₄O₄ requires 420.32396; EIMS (70 eV) m/z: 420 (M⁺, 2%), 391 (42), 265 (12), 237 (54), 149 (26), 111 (40) and 57 (100).

### Example 7

### Preparation of 5-hydroxy-4-(1-oxododecyl)-2-prenyl-2,6,6trimethyl-4-cyclohexene-1,3-dione

To a stirred suspension of K2CO₃ (240 mg) and the mono C dodecanoylated phloroglucinol (0.5 g), prepared as above in Example 1, in dry acetone (5 ml) was added prenyl bromide (0.27 ml), and the mixture refluxed for 5 h. The solvents were removed in vacuo to give an orange solid. Separation by column chromatography over silica gel eluting with with hexane with increasing amounts of dichloromethane then ethyl acetate gave firstly the tetraprenyl triketone, followed by a mixture of C and 0 mono-prenyl compounds (227 mg, 37%). Purification by crystallisation from dichloromethane gave a monoprenyl-phloroglucinol as a white crystalline solid (82 mg, 13%).

Sodium metal (0.15 g) was slowly added to methanol (3 ml) to form a solution. To this was added the monoprenyl-phloroglucinol (50 mg) followed by methyl iodide (3 ml). The solution was refluxed under nitrogen for 3 h. Addition of HCl (1 M) until just acidic, followed by extraction into ethyl acetate gave the crude product Purification by column chromatography over silica gel eluting with hexane with increasing amounts of dichloromethane gave 5-hydroxy-4(1-oxododecyl)2-prenyl-2,6,6trimethyl-4-cyclohexene-1,3-dione (45 mg, 82%) as a colourless oil: Si gel TLC (Hexane/Dichloromethane (50:50)), R_{F} 0.42 detection by UV light; UV (MeOH) λ max (log ε) 279 (3.9), and 238 (3.7) nm; IR (dry film) υ max 2926, 2854, 1721, 1672, 1666, 1580, 1564, 1549, and 1034 cm⁻¹; ¹H NMR (CDCl₃) 18.34 (1H s, OH), 4.73 (1H m, 2″), 2.95 (2H, m, C(2)H₂), 2.50 (2H, m, C(1″)H2), 1.62 (2H, m, C(3)H₂), 1.59, 1.56, 1.52, 1.47 (6H, s's, C(3″)Me₂), 1.44, 1.40, 1.39, 1.30, 1.28, 1.26 (9H, s's, C (3′)Me₂, C (5′)Me), 1.24 (16H, m) and 0.86 (3H, t, J 7 Hz, C(12)H₃) ppm; ¹³C NMR (CDCl₃) 210.2,209.8 (C-4′), 204.7, 204.6 (C-1), 197.7, 199.1 (C-2′), 196.5, 196.2 (C-6′), 137.1, 135.8 (C-3″), 117.5, 118.0 (C-2″),110.8, 110.1 (C-1′), 57.2, 61.0 (C-5′), 56.1, 51.9 (C-3′), 39.4,39.3 (p2), 38.9, 38.0 (C-3), 31.9, 29.6, 29.5, 29.4, 29.3, 26.2, 26.1, 25.9, 25.8, 25.3, 25.0, 22.6, 22.3, 22.2, 20.8, 20.3, 17.8, 17.7, 14.1 (C-12) ppm; Mass measurement, m/z Found, 418.30909; C₂₆H₄₂O₄ requires 418.31096; EIMS (70 eV) m/z: 418 (M⁺b, 10%), 375 (14), 350 (35), 322 (16), 293 (17), and 69 (100).

### Example 8

### Bioactivity Data

The antibacterial activities of compounds of Examples I to 7 were proved in biological assays.

Minimum inhibitory concentrations [MIC] were tested in Mueller Hinton [MH] broth. The human pathogen Methicillin Resistant Staphylococcus aureus (MRSA) (strain 1126) was provided by the University of Otago. Glass vials (30 mL) containing MH broth (10 mL) were inoculated from an actively growing culture and incubated overnight at 37° C. A standardized procedure to suspend each compound in MH broth was used. Each compound (5 mg) was dissolved in ethanol (1 mL). MH broth (9 mL) was then added to the ethanol containing the compound, resulting in stock solution concentrations of 500 μg /mL.

The MIC for each compound was determined by carrying out 3 replicate serial dilutions in Nunc® 48 well microtiter plates. To test the MICs, each compound stock solution was vigorously mixed before I mL was dispensed aseptically by pipette into 3 of the 6 first wells of a microtiter plate. MH broth (0.5 mL) was dispensed into all wells of the microtiter plate except the first row. Serial doubling dilutions were carried out through 12 wells (1½ microtiter plates). Mixing was carried out by repeated pipetting of 100 μL amounts. 500 μL was discarded from the last wells to conserve the intracellular compound concentration. Each well was inoculated with an aliquot (25 μL) of MRSA suspension diluted by 10⁻². All test and control microtiter plates were incubated at 37° C. and read after 48 h and 72 h. Results in Table 1 are the upper and lower limits of the MICs. The clinically proven antimicrobial agent vancomycin, used as a positive control, showed 3.1>MIC>1.6 μg/ml.

The compound of Example I was also tested, by similar methods to those above, against the food poisoning bacterium listera monocytogenes. It showed an MIC <3.9 μg/ml.

The compounds of Examples 1 to 7 were also tested against another Gram-positive bacterium, Bacillus subtilis (ATCC strain 19659), in disc diffusion assays. Solutions of compounds (60 μg/disc) were dried onto 6 mm diameter filter paper discs, which were then placed onto seeded agar Petri dishes and incubated (24 h). Activity showed as a zone of inhibition around the disc, with its width recorded from the edge of the disc in mm (Table 1). The positive control in this assay was the clinically proven antimicrobial agent chloramphenicol (30 μg/disc), which gave a 12 mm zone.

TABLE 1
Biological Assay Results for the Triketones

| Compound | | |
|---|---|---|
| of Example | MRSA activity¹ | BS activity² |
| 1 | 1.0 > MIC > 0.5 | 3 |
| 2 | 2.0 > MIC > 1.0 | 7 |
| 3 | 7.8 > MIC > 2.0 | 0 |
| 4 | 62.5 > MIC > 15.6 | 0 |
| 5 | 7.8 > MIC > 3.9 | 12 |
| 6 | 7.8 > MIC > 3.9 | 0 |
| 7 | 2.0 > MIC > 1.0 | 0 |

¹MRSA = Methicillin Resistant Staphylococcus aureus (University of Otago strain 1126). Activity given as upper and lower limits of Minimum Inhibitory Concentration in μg/ml.
²BS = Bacillus subtilis (ATCC Strain 19659). Activity given as width of zone of growth inhibition (mm) in a disc assay dosed at 60 μg/disc.

### Example 9

### Antibacterial Susceptibility

The compound of Example 1 also shows activity against other resistant bacteria.

Minimum inhibitory concentrations (MICs) were determined using a broth microdilution method. Bacterial isolates were inoculated into Todd-Hewitt broth (THB) and incubated at 37 C with shaking (200 rpm) for 18 h. Overnight cultures were diluted to an OD of 0.01 (at 595600 nm, 1 cm light path) to give a final inoculum for broth microdilution of OD 0.005. Doubling dilutions of the test compounds were prepared in THB in microtitre plates. Tween 80 (Sigma) was added at a final concentration of 14 % to enhance solubility. The range of concentrations tested (with doubling dilutions) was 16 μg/mL to 0.063 μg/mL. Microtitre plates were incubated at 37 ° C. for 18 h with gentle shaking. Plates were read using a microplate reader (Multiskan Ascent, Labsystems), measuring the OD at 595 nm. Minimum bactericidal concentrations (MBCs) were determined by placing 5 μL of culture from wells without growth onto THB plates and incubating at 37° C. for 18 h. The MBC was the lowest concentration without growth from the drop of culture. Experiments were performed in triplicate and the results of each experiment are shown in Table 2.

TABLE 2
Antibacterial Susceptibility Testing for the Compound of Example 1

| Strains Tested | MIC (μg/mL)* | | |
|---|---|---|---|
| Enterococcus faecalis | 1 | 0.5 | 1 |
| ATCC29212 | | | |
| Wild-type (fully susceptible) | | | |
| Enterococcus faecalis | 1 | 0.5 | 0.5 |
| CD2-49 | | | |
| Gentamicin resistant | | | |
| Enterococcus faecalis | 1 | 1 | 1 |
| 8A-25 | | | |
| Vancomycin resistant (VRE, | | | |
| vanA) | | | |
| E. faecium AR99/118 | 2 | 2 | 2 |
| Ampicillin resistant | | | |
| S. aureus ATCC25923 | 2 | 1 | 2 |
| Wild-type (fully susceptible) | | | |
| S. aureus OUM1127 | 1 | 1 | 1 |
| Erythromycin resistant | | | |
| S. aureus AS92/349 | 1 | 1 | 2 |
| Mupirocin resistant | | | |
| S. aureus ST94/1208 | 1 | 1 | 1 |
| Oxacillin/Gentamicin resistant | | | |
| (cMRSA) | | | |
| S. aureus OUM1126 | 1 | 1 | 1 |
| Oxacillin resistant (MRSA) | | | |
| S. aureus PC-3 | 2 | 2 | 2 |
| Vancomycin/Oxacillin resistant | | | |
| (GISA) | | | |

*Bacteriostatic at ≦2 μg/mL. Bacteriocidal at ≧8 μg/mL.

### Example 10

### Acute Toxicity

Compound I has been tested in mice for acute toxicity. Intra peritoneal (ip) injections (in cottonseed oil) at up to 400 mg/kg showed no toxic effects and as summarized in the protocol below:

A single trial was completed using twenty-five Swiss male mice (Mus musculus), weighing 20 g±2 g. Mice were selected by gender and weight, individually identified and randomly placed in 5 cages of 5 mice each. Mice were injected intraperitoneally with one of five levels of antibiotic [0 (control), 1, 2, 4 and 8 mg/ml]. Cottonseed oil was used as a carrier solution for the test substance. One mL samples were mixed before being injected using 1.0 ml syringes and 21G needles. Mice were observed continuously for 6 hours and any deaths or adverse symptoms recorded. This initial hour observation period was subsequently followed by another 18-hour monitoring period. Mice injected with the antibiotic showed no adverse toxic symptoms and no mouse deaths were recorded. This was also the case for mice injected solely with cottonseed oil. This is in direct contrast to weekly calibration assays using the toxic control saxitoxin dihydrochloride.

Although the invention has been described by way of example, it should be appreciated that variations and modifications may be made without departing from the scope of the claims. Furthermore, where known equivalents exist to specific features, such equivalents are incorporated as if specifically referred to in this specification.

1-13. (canceled)

14. A compound of Formula (1): 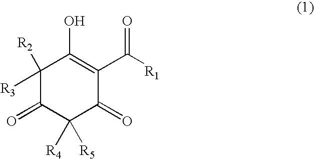
where R₁ is a group selected from alkenyl, alkynyl and C₆-C₂₀ alkyl, each of which may be substituted with one or more of the groups selected from hydroxy, halogen, amino, alkylamino, di-alkylamino, haloalkyl, nitro, cyano, —SO₃H and triphenylphosphine;
or R₁ is a group selected from aryl, C₅-C₈ cycloalkyl, (C₁-C₂₀ alkyl)cycloalkyl and (C₁-C₂₀ alkyl)aryl, each of which may be substituted with one or more of the groups selected from hydroxy, halogen, amino, alkylamino, di-alkylamino, haloalkyl, nitro, cyano, —SO₃H, triphenylphosphine, alkyl, alkenyl, and alkynyl; provided that R₁ is not —CH₂CH₂phenyl; and
R₂ to R₅ are each independently alkyl, alkenyl or alkynyl groups, each of which may be substituted with one or more of the groups selected from hydroxy, halogen, amino, alkylamino, di-alkylamino, haloalkyl, nitro, cyano, —SO₃H and triphenylphosphine;
or R₂ to R₅ are each independently aryl or acyl groups, each of which may be substituted with one or more of the groups selected from hydroxy, halogen, amino, alkylamino, di-alkylamino, haloalkyl, nitro, cyano, —SO₃H, triphenylphosphine, alkyl, alkenyl and alkynyl; or a salt thereof, or a metal complex thereof, or any tautomeric form thereof.

15. A compound as claimed in claim 14 wherein R₂ to R₅ are each independently alkyl or alkenyl groups.

16. A compound as claimed in claim 14 wherein R₂ to R₅ are all methyl groups.

17. A compound as claimed in claim 14 wherein one or more of R₂ to R₅ is a prenyl group.

18. A compound as claimed in claim 14 wherein R₁ is C₆-C₂₀ straight chain alkyl or (C₁-C₂₀ alkyl)phenyl, provided that R₁ is not —CH₂CH₂phenyl.

19. A compound as claimed in claim 14 wherein R₁ is C₁₀-C₁₆ straight chain alkyl.

20. A compound as claimed in claim 14 which is:
5-hydroxy-4-(1-oxododecyl)-2,2,6,6-tetramethyl-4-cyclohexene-1,3-dione;
5-hydroxy-4-(1-oxodecyl)-2,2,6,6-tetramethyl-4-cyclohexene-1,3-dione;
5-hydroxy-4-(1-oxohexadecyl)-2,2,6,6-tetramethyl-4-cyclohexene-1,3-dione;
5-hydroxy-4-(1-oxomethylcyclohexyl)-2,2,6,6-tetramethyl-4-cyclohexene-1,3-dione;
5-hydroxy-4-(1-oxopropylcyclohexl)-2,2,6,6-tetramethyl-4-cyclohexene-1,3-dione;
5-hydroxy-4-(1-oxododecyl)-2,2,6,6-tetraethyl-4-cyclohexene-1,3-dione; or
5-hydroxy-4-(1-oxododecyl)-2-prenyl-2,6,6-trimethyl-4-cyclohexene-1,3-dione.

21. A pharmaceutical composition containing a compound as claimed in claim 14 together with a pharmaceutically acceptable carrier.

22. A pharmaceutical composition containing a compound as claimed in claim 20 together with a pharmaceutically acceptable carrier

23. An antibacterial agent containing a compound as claimed in claim 14.

24. An antibacterial agent containing a compound as claimed in claim 20.

25. The use of a compound as claimed in claim 14 in the manufacture of an antibacterial agent.

26. The use of a compound as claimed in claim 20 in the manufacture of an antibacterial agent.

27. A process for preparing a compound as claimed in claim 14, including the steps of:
(a) reacting phloroglucinol with a carboxylic acid of the formula R₁—COOH, or equivalent acid halide, nitrite or anhydride, where R₁ is as defined in claim 14, to give a substituted phloroglucinol of the formula 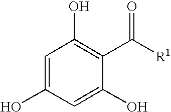
(b) reacting the substituted phloroglucinol with one or more compounds of the formula AX where A is R₂, R₃, R₄ or R₅, each of which is as defined in claim 14, and X is a halogen atom.

28. A method of treatment or prevention of a bacterial infection in a human or other animal comprising administering to the human or other animal a therapeutically effective amount of a compound as claimed in claim 14.

29. A method of treatment or prevention of a bacterial infection in a human or other animal comprising administering to the human or other animal a therapeutically effective amount of a compound as claimed in claim 20.

30. The method as claimed in claim 28, where the bacterial infection is caused by Staphylococcus aureus, Methicillin Resistant Staphylococcus aureus, Erythromycin Resistant Staphylococcus aureus, Mupirocin Resistant Staphylococcus aureus, Oxacillin/Gentamicin Resistant Staphylococcus aureus, Vancomycin/Oxacillin Resistant Staphylococcus aureus, Bacillus subtilis, Enterococcus faecalis, Gentamicin Resistant Enterococcus faecalis, Vancomycin Resistant Enterococcus faecalis, or Ampicillin Resistant Enterococcus faecalis.
